(19) United States
(12) Patent Application Publication
(10) Pub. No.: US 2019/0134268 A1
(43) Pub. Date: May 9, 2019

(54) MEMBRANOUS TISSUE WITH EVENLY SPACED ELEVATED PROJECTIONS ON ONE SIDE AND CONCAVE DEPRESSIONS ON THE OTHER SIDE METHOD AND USE

(71) Applicant: Theodore I. Malinin, Key Biscayne, FL (US)

(72) Inventor: Theodore I. Malinin, Key Biscayne, FL (US)

(21) Appl. No.: 15/803,967

(22) Filed: Nov. 6, 2017

(51) Int. Cl. A61L 27/36; A61L 27/60

(52) U.S. Cl. CPC A61L 27/3691; A61L 27/60; A61L 27/3695; A61L 27/362; A61L 2400/18; A61L 27/3604; A61L 2430/20; A61L 2430/40; A61L 2430/34; A61L 27/3625

(57) ABSTRACT

The present invention discloses the method of preparation and use of soft tissue membranous structures into slip resistant membranes with regularly spaced surface projections on one side and concave depressions on the other side with perforations or without perforations which enhance vascular ingrowth and tissue incorporation.

### TECHNICAL FIELD

This invention is related to membranous tissue with evenly spaced elevated projections on one side and concave depressions on the other side.

### BACKGROUND OF THE INVENTION

Smooth membranous tissue allografts are widely used clinically in promoting healing and tissue reconstruction. These membranes include dermis, placental membranes, fasciae, dura mater and pericardium. Some of those membranes are used fresh or frozen, but a large number are preserved by drying, often freeze-drying. The latter has an advantage of prolonged storage at ambient temperatures. Dermis allografts represent high quality regenerative material which exhibit multidirectional strength and adaptability to surface contours. These are usually freeze-dried and packaged aseptically. Dermis allografts enjoy a long history of effective clinical results Amnion and chorion membranes have gained in recent popularity, although their use dates back to the beginning of the last century.

These allografts are used for volume enhancement and provide space for angiogenesis and tissue remodeling. These grafts have been also effective in the stabilization and healing of recalcitrant wounds. These allografts are biocompatible and are incorporated into the tissues of the host during the healing process. However, incorporation of the graft is delayed because blood vessels, during angiogenesis, must overcome the solid barrier of decellularized dermis or cellular and intact tissues. The other problem with smooth membranous tissue allografts is slippage when positioned onto a wound site or a defect being repaired.

Placental membranes, amnion and chorion, have been used in surgical applications for over a hundred years. Recently these have been retooled and made available to treat acute and chronic wounds, prevent adhesions, reduce inflammation and decrease pain. These membranes contain growth factors as well as anti-inflammatory factors Amnion as well as chorion are furnished dehydrated, usually freeze-dried. Other smooth membranes such as pericardium, fasciae and dura mater have their own clinical applications.

As has been mentioned, all of the above listed membrane preparations have smooth surfaces. This leads to sliding of the allograft membrane once they are placed in a particular defect. Smooth surfaces likewise are not conducive to vascular ingrowth.

The present invention overcomes both of these difficulties by providing anchorage and channels for blood vessel ingrowth.

### SUMMARY OF THE INVENTION

The invention provides for converting smooth soft tissue membrane allografts into textured, slip-resistant membranes which enhance vascular ingrowth and tissue incorporation. The present invention describes the method of preparing textured soft tissue membrane allografts by freeze-drying these on metallic grids with perforations. Tissues are forced into metallic perforations, and form permanent elevated structures on the under-surface of the membrane being dried. The membrane can be placed on the perforated metal plate or structure with either side facing up or down depending on which way the depressions are desired to be formed.

The present invention overcomes these deficiencies making the membranes with regular protrusions easy to retain in place. Vascular ingrowth is likewise enhanced by protrusions and indentations. In addition, the ends of the regular protrusions can be cut or punctured, thus rendering the allografts into perforated structures.

A method of making smooth two-sided wetted or non-dried biological membranes into three-dimensional membrane structures with regularly or irregularly spaced surface concave depressions on a first side and projections on the opposite second side is described as follows. The method has the steps of: acquiring a smooth two sided wetted or non-dried membrane for drying; placing the smooth wetted or non-dried membrane on a support surface of a perforated plate, the perforated plate having a plurality of holes or depressions on the support surface of the plate for forming depressions on a first side of the membrane and projections on an opposite second side adjacent the support surface; and drying the smooth wetted or non-dried membrane on the perforated plate wherein the smooth wetted or non-dried membrane locally sags or sinks into the perforations forming depressions on the first side and corresponding projections on the opposite second side of the membrane converting the membrane into a three-dimensional structure when dried. The projections or depressions form a non-slip surface when implanted.

The method may have the step of perforating at least one or more of the surface projections on the second side and concave depressions on the first side in the membranes to form open perforations at an end of the at least one or more projections of the three-dimensional membrane structure.

In one embodiment, the smooth two-sided wetted or non-dried membrane is freeze-dried on perforated plates and retained in place by compressing the membrane with solid plates or under vacuum. The membranes to be freeze-dried are preferably covered with perforated cellophane.

The biological membrane material can be one of dermis, full thickness skin, amniotic membrane, pericardium, dura mater or fascia. The dermis, full-thickness skin, amniotic membrane, fascia, pericardium, dura mater or other membranes can be human or animal. The dermis can be intact or decellularized.

The step of perforating the at least one or more projections and concave depressions provides a fluid pathway for vascularization when implanted.

A three-dimensional membrane structure has a smooth two-sided biological membrane with regular or irregularly spaced surface concave depressions on a first side and projections on an opposite second side. The three-dimensional membrane structure can have at least one or more of the surface projections on the second side and concave depressions on the first side in the membranes form open perforations at an end of the at least one or more projections of the three-dimensional membrane structure. Preferably, the three-dimensional membrane is a dried membrane, more preferably, the dried membrane can be freeze-dried. The biological membrane material is one of dermis, full thickness skin, amniotic membrane, pericardium, dura mater or fascia, and wherein the dermis, full-thickness skin, amniotic membrane, fascia, pericardium, dura mater or other membranes can be human or animal also the dermis can be intact or decellularized.

In an alternative embodiment, a combination of the projections and concave depressions may be used on a same side or surface of the membrane such that the resultant three-dimensional structures have traction and flow features from both surfaces.

### BRIEF DESCRIPTION OF THE DRAWINGS

The invention will be described by way of example and with reference to the accompanying drawings in which:

FIG. 1 is a photograph of a metal plate with perforations on which the membrane is placed, frozen and then freeze-dried. The perforations are placed in a predetermined geometric pattern, but this can vary.

FIG. 2 provides a comparison between dermis freeze-dried on a smooth metal plate (right) and on a perforated metal plate (left). The projections on one side and concave depressions on the other side are produced by tissue being forced into the metal plate perforations by the vacuum in the freeze-drying chamber.

FIG. 2A is a cross-section showing a wetted membrane on the perforated plate between two smooth plates.

FIG. 3 shows dermis after being freeze-dried on the plate shown in FIG. 1.

FIG. 4 is a photograph of freeze-dried dermis laid upside down on the metal plate on which it was freeze-dried. The protrusions are concave on the inside.

FIG. 5 provides a close-up view of the protrusions with perforations of dermis having been freeze-dried on the perforated metal plate.

FIG. 6 shows a membrane with a combination of protrusions and concave depressions on each surface side.

### DETAILED DESCRIPTION OF THE INVENTION

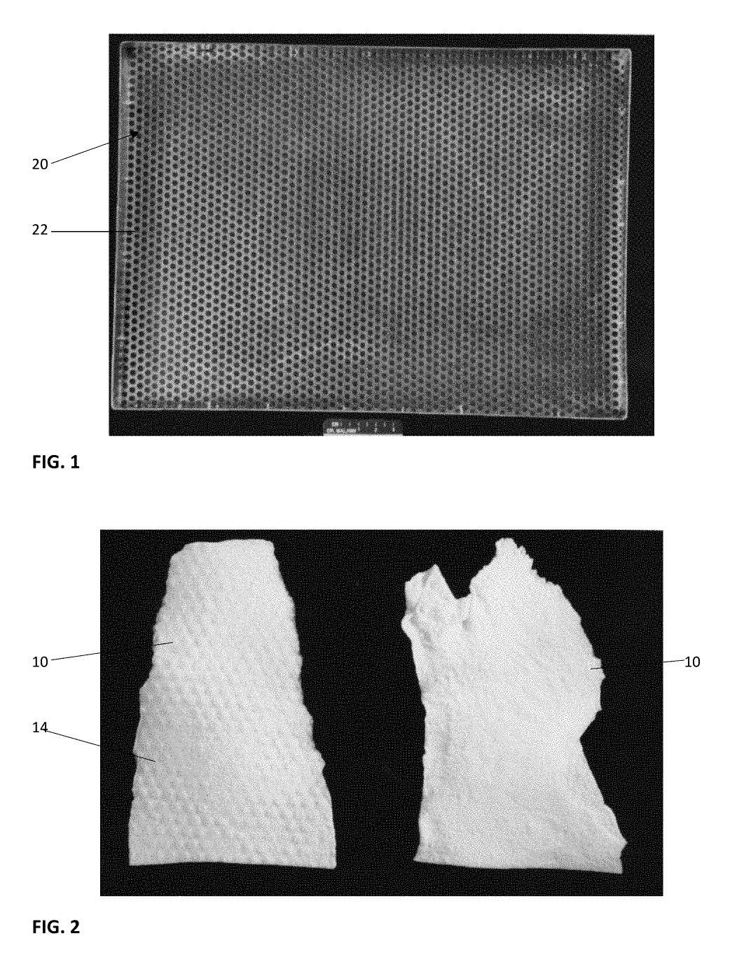

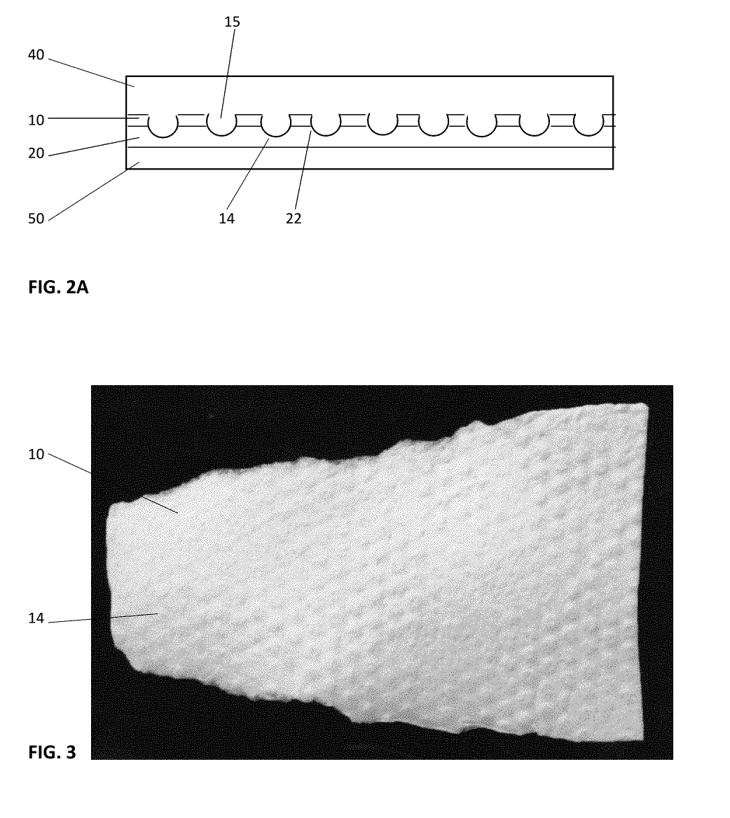

The invention is directed to the finding that when membranous tissue 10, when fresh is placed and frozen on a perforated metal plate 20, shown in FIG. 1, in the chamber of a freeze dryer and then freeze-dried, the smooth structure is converted into a structure with regularly spaced projections 14 on one side and concave depressions 15 on the other side on the surface. The stainless-steel plate 20 shown in FIG. 1 with perforations 22 on which the membrane 10 is frozen and freeze-dried can be made of a variety of metals. This biomechanical change is irreversible; freeze-dried membrane 10 will retain its shape on rehydration, illustrated in FIG. 2.

With reference to FIG. 2, on the left side is pictured the membrane 10, in this picture dermis freeze-dried on the plate 20 shown in FIG. 1. On the right side of FIG. 2, dermis freeze dried on a solid metal plate is shown. The wetted or non-dried membrane is shown in FIG. 2A on the plate 20 and pressed between two smooth plates 40, 50.

Skin dermis is prepared by separating it from the epidermis by a dermatome, any cutting devise or by chemical dissolution of the epidermis. Dermis (fresh) is placed on a perforated metal grid or plate 20 which is inserted into the chamber of a freeze drier at room temperature. The shelf cooling is turned on concomitantly with turning on the vacuum in the chamber. The shelf is cooled to at least 15° C. and the membrane 10 placed on it is frozen. Freeze-drying commences after the membrane 10 is frozen. The vacuum in the chamber is maintained at 100 millitorr or below. After freeze-drying is completed with residual moisture in the membrane 10 reduced to about 15%, the chamber is returned to atmospheric pressure, and the membrane 10 whose shape was altered is removed, illustrated in FIG. 3. Membranous tissue such as fascia lata, dura mater or pericardium are treated intact in an identical manner

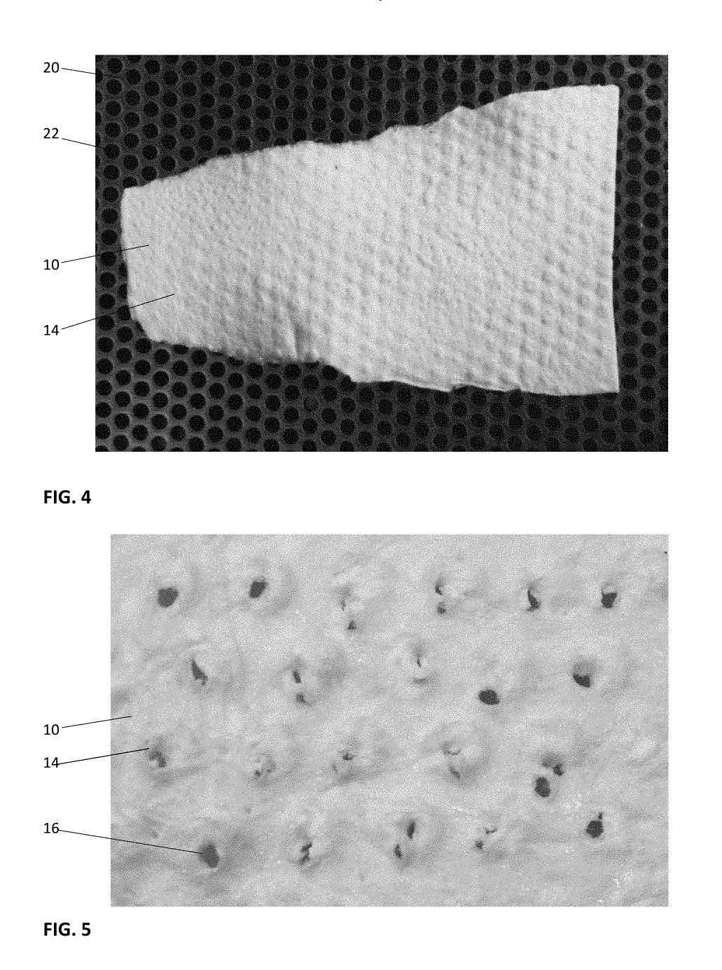

In general, amniotic membrane is produced by drying not freeze-drying, fresh amniotic membrane. The latter can be used as a substitute membrane for a membrane tissue in a living body. The amniotic membrane is useful as a medical substitute membrane such a dura meter, pericardium, pleura, peritoneum and for coverage of non-healing wounds, such as those of the extremities associated venous stasis. The method used for drying amniotic membrane involves heating the amniotic membrane under reduced pressure to 50° C. (U.S. Pat. No. 8,414,929). The heating is produced by a far-infrared heater and a microwave irradiator. The fragmentation of the amniotic membrane, said to occur with conventional freeze-drying, is prevented by repeating pressure reducing step. In the present invention potential convenience freeze-drying damage is avoided by rapidly freezing amniotic membrane on perforated metallic plate before placing it in the freeze-drying chamber and reducing the pressure in the freeze-drying chamber to room millitorr or below. In another embodiment, the amniotic membrane is covered with perforated cellophane to keep it flat during the freeze-drying process. In still another embodiment, the amniotic membrane 10 is covered with a heavy plate, metal, plastic, Teflon or any other material to keep it flat. Adequate drying is achieved through holes 22 of the perforated metal plate 20. Preferably the entire process is performed aseptically, but if aseptic technique is not used the membrane 10 can be secondarily sterilized by irradiation, ethylene-oxide sterilization, peracetic acid or any other generally acceptable methods. Freeze-dried amniotic membrane with artificial regularly spaced bumps, produced by the described technique can be stored in sterile packages. Since cellular components are left intact in the amniotic membrane produced by the described method, rehydrated amniotic membrane would maintain biologic properties similar to those of fresh amnion. Irregular surface would prevent slippage of the implant and facilitate vascular ingrowth. With reference to FIG. 4, the freeze-dried dermis 10 is laid upside down on the metal plate 20 on which it was freeze-dried to show the protrusions 14 which are concave depressions 15 on the opposite side.

Perforations 16 in the projections 14 produced by the above-described method would enhance vascular ingrowth and accelerate incorporation of the membranous grafts. These can be made by pressing the membrane 20 with a solid plate 40, 50 to maintain it in place on the perforated metal plate 20, and cutting off the ends of the cover with a sharp blade. In another embodiment the holes in the cover can be made by needles superimposed over the perforations 22 in the perforated metal plate 20.

FIG. 5 shows a close up view of the protrusions 15 with perforations 16. These facilitate vascular ingrowth and incorporation of the membranous allograft. Centers of the projections 14 on one side and concave depressions 15 on the other side are pitted.

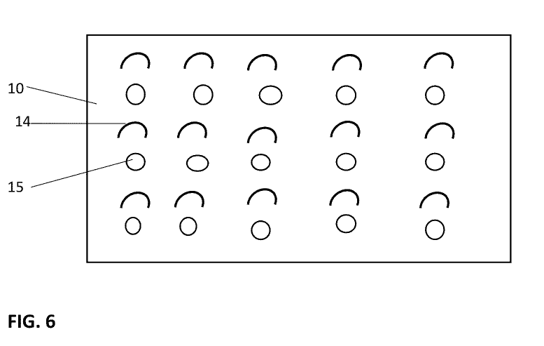

With reference to FIG. 6, an alternative embodiment shows a combination of the projections 14 and concave depressions 15 may be made on a same side or surface of the membrane 10 such that the resultant three-dimensional structures have traction and flow features from both surfaces.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

What is claimed is:

1. A method of making smooth two-sided wetted or non-dried biological membranes into three-dimensional membrane structures with regularly or irregularly spaced surface concave depressions on an at least first side and projections on at least the opposite second side, the method comprising the steps of:
acquiring a smooth two sided wetted or non-dried membrane for drying;
placing the smooth wetted or non-dried membrane on a support surface of a perforated plate, the perforated plate having a plurality of holes or depressions on the support surface of the plate for forming depressions on a first side of the membrane and projections on an opposite second side adjacent the support surface; and
drying the smooth wetted or non-dried membrane on the perforated plate wherein the smooth wetted or non-dried membrane locally sags or sinks into the perforations forming depressions on the first side and corresponding projections on the opposite second side of the membrane converting the membrane into a three-dimensional structure when dried.

2. The method of claim 1 further comprises the step of perforating at least one or more of the surface projections on the second side and concave depressions on the first side in the membranes to form open perforations at an end of the at least one or more projections of the three-dimensional membrane structure.

3. The method of claim 1 wherein the smooth two-sided wetted or non-dried membrane is freeze-dried on perforated plates and retained in place by compressing the membrane with solid plates or under vacuum.

4. The method of claim 1 wherein either prior to drying or post drying the three-dimensional structure has projections formed on the first side with the concave depressions so the membrane has the first side with a configuration of projections and concave depressions and the second side similarly has a combination of concave depressions and projections.

5. The method of claim 3 wherein the membranes to be freeze-dried are covered with perforated cellophane.

6. The method of claim 1 wherein the biological membrane material is one of dermis, full thickness skin, amniotic membrane, pericardium, dura mater or fascia.

7. The method of claim 6 wherein the dermis can be intact or decellularized.

8. The method of claim 6 wherein the dermis, full-thickness skin, amniotic membrane, fascia, pericardium, dura mater or other membranes can be human or animal

9. The method of claim 2 wherein the step of perforating the at least one or more projections and concave depressions provides a fluid pathway for vascularization when implanted.

10. The method of claim 1 wherein the projections or depressions form a non-slip surface when implanted.

11. A three-dimensional membrane structure comprises:
a smooth two-sided biological membrane with regular or irregularly spaced surface concave depressions on a first side and projections on an opposite second side.

12. The three-dimensional membrane structure of claim 11 wherein at least one or more of the surface projections on the second side and concave depressions on the first side in the membranes form open perforations at an end of the at least one or more projections of the three-dimensional membrane structure.

13. The three-dimensional membrane structure of claim 11 wherein the three-dimensional membrane is a dried membrane.

14. The three-dimensional membrane structure of claim 13 wherein the dried membrane has been freeze-dried.

15. The three-dimensional membrane structure of claim 11 wherein the biological membrane material is one of dermis, full thickness skin, amniotic membrane, pericardium, dura mater or fascia.

16. The three-dimensional membrane structure of claim 15 wherein the dermis can be intact or decellularized.

17. The three-dimensional membrane structure of claim 15 wherein the dermis, full-thickness skin, amniotic membrane, fascia, pericardium, dura mater or other membranes can be human or animal.
